(19) United States
(12) Patent Application Publication
(10) Pub. No.: US 2012/0207821 A1
(43) Pub. Date: Aug. 16, 2012

(54) Liposomal formulation and use thereof

(76) Inventors: Nahid Ali, Kolkata (IN); Jayeeta Ghose, Kolkata (IN); Swati P. Bhowmick, Kolkata (IN)

(21) Appl. No.: 13/506,411

(22) Filed: Apr. 17, 2012

Related U.S. Application Data
(63) Continuation of application No. 11/439,381, filed on May 23, 2006.

(30) Foreign Application Priority Data
May 25, 2005 (IN) 1339/DEL/2005

(51) Int. Cl. A61K 9/127; A61P 33/02; B01J 13/04

(52) U.S. Cl. 424/450; 264/4.1

(57) ABSTRACT

Cationic liposome encapsulated antimonial drugs formulations are provided. The drug-loaded liposome have enhanced efficacy as antileismanial agents and provide improved therapeutic index as compared to the minimal dose of free drug.

This application claims the right of priority under 35 U.S.C. §119(a)-(d) to Indian Patent Application No. 1339/DEL/2005, filed May 25, 2005 and the text of application 1339/DEL/2005 is hereby incorporated by reference in its entirety.

### FIELD OF INVENTION

The present invention relates to a cationic liposomal formulation useful as a leishmanicidal agent. More particularly, it relates to the use of liposomal formulation in the treatment of kala azar. Further, it also relates to a pharmaceutical composition useful for the treatment of Kala azar in a subject. More specifically, it relates to a method of treating the kala azar in a subject. Further, the present invention also relates to a method for the preparation of liposomal formulation.

### BACKGROUND AND PRIOR ART OF THE INVENTION

Protozoan parasites of the genus Leishmania cause a spectrum of diseases ranging from diffused cutaneous lesions (Diffused cutaneous leishmaniasis [DCL]), Local cutaneous leishmaniasis (LCL), mucocutaneous lesions (Espundia), to the more severe form of visceralized disease (Visceral Leishmaniasis [VL] or Kala-azar) in addition to the comparatively rare and illusive post kala-azar dermal leishmaniasis (PKDL). Sandflies of the genera Phlebotomus and Lutzomia act as vectors of all the diseases caused by Leishmania parasites and transmission modes vary from anthroponotic to zoonotic, with a variety of mammalian animals implicated as reservoirs.

Visceral Leishmaniasis or Kala-azar is characteristically symptomized by fever, hepatosplenomegaly (Splenomegaly greater than hepatomegaly as opposed to malaria), pancytopenia, and progressive deterioration of the health of the host. Occasionally, kala-azar is followed by a dermal manifestation of PKDL, which, incidentally, never visceralizes. Kala-azar and PKDL are caused by Leishmania donovani in India, Leishmania infantum in Africa and Leishmania chagasi. Widespread papules or nodules in the skin all over characterize DCL while LCL typically exhibits localized lesions. Mucocutaneous leishmaniasis or espundia is more common in Latin America and the disease causes severe ulceration in and around the linings of the naso-pharangeal region. Cutaneous leishmaniasis (oriental sore) is caused by either of the etiological agents like L. major, L. tropica and L. aethiopicain in Old World and L. guyanensis, L. panamensis and L. mexicana in New World.

Until recently, the entire spectrum of leishmaniases in all its forms was absolutely curable with antimony compounds. Despite extended treatment regimens, parenteral administration and toxic side effects, the pentavalent antimonials still remains the cornerstone of treatment for all forms of leishmaniasis for more than sixty years (Berman et. al., Am. J. Trop. Med. Hyg, 46, 296-306, 1992 and Thakur et. al., Ann. Trop. Med. Parasitol 92, 561-569, 1998). Pentavalent antimonials are complexed to gluconic acid to form sodium stibogluconate (Pentostam) or meglumine antimoniate (Glucantime). It is conceivable that the mechanism of Pentostam is via the small amount (0.5%) that binds to parasite nucleic acid or via binding to small molecular weight (<8000 Da) parasite components. But still the exact mechanism of action needs to be elucidated.

However, the steady erosion in the response to treatment with sodium antimony gluconate has been the most recent outcome of the kala-azar epidemic. This necessitates the use of more toxic second line drugs, amphotericin-B and pentamidine (Jha., Trans. R. Soc. Trop. Med. Hyg, 77, 167-170, 1983 and Jha et. al., Am. J. Trop. Med. Hyg, 52, 536-538, 1995 and Ali et. al., Ann. Trop. Med. Parasitol, 92, 151-158, 1998). Nowadays, several lipid-based formulations of amphotericn-B are being used with reduced toxicities (Bryceson et al., Clin. Infect. Dis, 22, 938-943, 1996 and Ali et al., Antimicrob. Agents. Chemother, 44, 1739-1742, 2000 and Murray et al., Ann. Intern. Med, 127, 133-137, 1997). Some proposals were available for the formulation of the antimonial drugs into liposome (Alving et al., Proc. Natl. Acad. Sci. USA, 75, 2959-2963, 1978 and Black et al., Trans. R. Soc. Trop. Med. Hyg, 71, 550-552, 1977 and Peters et. al., Nature, 272, 55-56, 1978). Such liposomal formulations are of interest because liposome encapsulated SAG was found to be 200-700 times more active than free SAG. A patent filed by Dr. L. S. Rao (Rao, U.S. Pat. No. 4,594,241) also demonstrated an effective antileishmanial liposomal sodium antimony gluconate formulation. However, the liposomal formulation that have been proposed so far suffer from disadvantages of optimal dose of the antimonial drug need to be incorporated and/or relatively high leakage rates of the antimonial drugs from the encapsulated aqueous phase in to the continuous phase on storage. Moreover, such formulations are unable to remove parasites present in deep-seated organs like spleen and bone marrow.

Liposomes are spherical vesicles, with particle size ranging from 30 nm to several micrometers, consisting of one or more lipid bilayers surrounding aqueous spaces. (Vemuri, et. al., Pharm. Acta. Helv, 70: 95-111, 1995). Hydrophilic drugs can be encapsulated in the initial aqueous compartment, whereas hydrophobic drugs may be bind to or incorporated in the lipid bilayers completely closed bilayer membranes containing an entrapped aqueous volume. Liposomes may be unilamellar vesicles (possessing a single membrane bilayer) or multilamellar vesicles (onion like structure characterized by multiple membrane bilayers, each separated from the next by an aqueous layer). The structure of the membrane bilayer is such that the hydrophobic (nonpolar) “tails” of the lipid orient towards the center of the bilayer while the hydrophilic (polar) “heads” orient towards the aqueous phase (Weiner et al, U.S. Pat. No. 6,759,057). The original liposome preparation of Bangham et al. (J. Mol. Biol. 13, 238-252, 1965) results in the formulation of multilamellar vesicles. It involves suspending phospholipids in an organic solvent, which is then evaporated to dryness leaving a phospholipid film on the round-bottomed reaction vessel. Subsequently, an appropriate amount of aqueous phase is added and the mixture is allowed to “swell” and dispersed by mechanical means, leading to the formation of MLV. This technique provides the basis for the development of the small-sonicated unilamellar vesicles described by Papahadjopoulas et al. (Biochim. Biophys. Acta. 135, 624-638, 1976) and large unilamellar vesicles.

Liposome can be used as drug delivery system that helps to increase the therapeutic index of the injected drugs by increasing the concentration of drug at the site of infection and thereby reducing the amount of drug required to eradicate the disease. In such a liposome-drug delivery system, the medicament is entrapped into the liposome and then administered to the patient to be treated. For example, see Rahman et. al., U.S. Pat. No. 3,993,754; Sears, U.S. Pat. No. 4,145,410; Papahadjopoulos et. al., U.S. Pat. No. 4,235,871.

### OBJECTS OF THE INVENTION

The main object of the present invention is to provide a liposomal formulation useful as a leishmanicidal agent.

Another object of the present invention is to provide the use of liposomal formulation in the treatment of kala azar.

Further another object of the present invention is to provide a liposomal formulations encapsulating sodium antimony gluconate which can target deep hidden parasites and also therapeutically active against drug resistant strain.

Yet another object of the present invention is to provide a liposomal formulation wherein cationic liposomes themselves have leishmanicidal activity and on encapsulating sodium antimony gluconate into them further improve their therapeutic potentiality.

Still another object of the present invention is to provide a pharmaceutical composition useful for the treatment of Kala azar in a subject.

Still another object of the present invention is to provide a method of treating the kala azar in a subject.

Still another object of the present invention is to provide a method for the preparation of liposomal formulation.

### SUMMARY OF THE INVENTION

The present invention deals with the liposomal formulation, method for the preparation and the use thereof which include liposome comprising various cationic lipids associated with neutral lipids and sodium antimony gluconate wherein cationic liposomes themselves have leishmanicidal activity and on encapsulating sodium antimony gluconate into them further improve their therapeutic potentiality. It also relates to a pharmaceutical composition useful for the treatment of Kala azar and a method of treating the kala azar in a subject.

### DETAILED DESCRIPTION OF THE INVENTION

Accordingly, the present invention provides a liposomal formulation useful as a leishmanicidal agent, wherein the said formulation comprising the therapeutically effective amount of antileishmanial antimonial drugs encapsulated in sub optimal dose of cationic liposome wherein the ratio of the lipid to drug is in the range of 63:1 to 44:1.

In an embodiment of the present invention, the antileishmanial antimonial drugs used is selected from the group comprising of pentavalent antimonial drugs, trivalent antimonial drugs etc.

In another embodiment of the present invention, the said liposome comprises a neutral lipid and a cationic lipid in a molar ratio of 7:2, respectively.

Further, in another embodiment of the present invention, the neutral lipid used is selected from the group consisting of phosphatidylcholine of type X-E, egg phosphatidylcholine and hydrogenated egg phosphatidylcholine.

Yet, in another embodiment of the present invention, the said phosphatidylcholine is selected from a group comprising of distearoylphosphatidylcholine, hydrogenated soy phosphotidylcholine, phoshatidylglycerol, diaurylphosphatidylglycerol, dipalmitoylphosphatidylglycerol, distearoylposphatidylglycerol, soy phosphatidylcholine, dimyristoylphosphatidylcholine, dipalmitoylphosphatidylcholine, dioleoylphosphatidylethanolamine and dimyristoylphosphatidylglycerol, dilaurylphosphatidylglycerol.

Still in another embodiment of the present invention, the said egg phosphatidylcholine is selected from a group comprising of distearoylphosphatidylcholine, hydrogenated soy phosphotidylcholine, phoshatidylglycerol, diaurylphosphatidylglycerol, dipalmitoylphosphatidylglycerol, distearoylposphatidylglycerol, soy phosphatidylcholine, dimyristoylphosphatidylcholine, dipalmitoylphosphatidylcholine, dioleoylphosphatidylethanolamine and dimyristoylphosphatidylglycerol, dilaurylphosphatidylglycerol.

Still, in another embodiment of the present invention, the said hydrogenated egg phosphatidylcholine is selected from a group consisting of distearoylphosphatidylcholine, hydrogenated soy phosphotidylcholine, phoshatidylglycerol, diaurylphosphatidylglycerol, dipalmitoylphosphatidylglycerol, distearoylposphatidylglycerol, soy phosphatidylcholine, dimyristoylphosphatidylcholine, dipalmitoylphosphatidylcholine, dioleoylphosphatidylethanolamine and dimyristoylphosphatidylglycerol, dilaurylphosphatidylglycerol.

Still, in another embodiment of the present invention, the cationic lipid used is selected from the group consisting of octadecylamine, dimethyldioctadecylammoniumbromide, cetryltrimethylammoniumbromide and dodecyltrimethylammoniumbromide.

Still, in another embodiment of the present invention, the said octadecylamine is selected from a group of cationic lipids comprising of dioleoyltrimethylammoniumpropane and dimyristoyltrimethylammoniumpropane.

Still, in another embodiment of the present invention, the said dimethyldioctadecylammoniumbromide is selected from a group comprising of dioleoyltrimethylammoniumpropane and dimyristoyltrimethylammoniumpropane.

Still, in another embodiment of the present invention, the said cetryltrimethylammoniumbromide is selected from a group comprising of dioleoyltrimethylammoniumpropane and dimyristoyltrimethylammoniumpropane.

Still, in another embodiment of the present invention, the said odecyltrimethylammoniumbromide is selected from a group comprising of dioleoyltrimethylammoniumpropane and dimyristoyltrimethylammoniumpropane.

Still, in another embodiment of the present invention, the said liposome is selected from the group consisting of multilamellar vesicle, unilamellar vesicle, dehydrated-rehydrated vesicle, reverse-phase evaporation vesicle.

Still, in another embodiment of the present invention, the said liposome is suspended in pharmaceutically acceptable carriers selected from the group consisting of salts such as sodium chloride, sodium dihydrogen phosphate and disodium hydrogen phosphate in a concentration such that the osmolarity of the continuous aqueous phase is same that of the human blood.

Still, in another embodiment of the present invention, the said formulation is stable at a pH of 7-7.8 whereby the leakage rate of initially encapsulated said antimonial drugs are less than 50% by weight after storage for 4 weeks, at 4° C., from the day of encapsulation.

Further, it also provides the use of liposomal formulation in the treatment of kala azar.

The present invention also provides a pharmaceutical composition useful for the treatment of Kala azar in a subject, wherein the said composition the said composition comprising the therapeutically effective amount of a liposomal formulation suspended in one or more commercially available pharmaceutically acceptable carriers.

In an embodiment of the present invention, the pharmaceutically acceptable carriers used are selected from the group consisting of salts such as sodium chloride, sodium dihydrogen phosphate and disodium hydrogen phosphate in a concentration such that the osmolarity of the continuous aqueous phase is same that of the human blood.

In another embodiment of the present invention, the dosage of the said composition is administered at a unit dose of at least 0.015 g/kg of SAG entrapped in 1.0-1.1 g/kg PC-SA liposome.

Further, in another embodiment of the present invention, the administration route used is selected from the group comprising of intravenous, intramuscular, intralesional etc.

Further, the present invention also provides a method of treating the kala azar in a subject, wherein the said method comprising the step of administering to the subject a pharmaceutical composition comprising the therapeutically effective amount of liposomal formulation suspended in one or more pharmaceutically acceptable carriers.

In an embodiment of the present invention, the said method comprises the step of administering to the subject a pharmaceutical composition.

In another embodiment of the present invention, the pharmaceutically acceptable carriers used are selected from the group consisting of salts such as sodium chloride, sodium dihydrogen phosphate and disodium hydrogen phosphate in a concentration such that the osmolarity of the continuous aqueous phase is same that of the human blood.

In another embodiment of the present invention, the dosage of the said composition administered is at a unit dose of at least 0.015 g/kg of SAG entrapped in 1.0-1.1 g/kg PC-SA liposome.

Further, in another embodiment of the present invention, the administration route used is selected from the group comprising of intravenous, intramuscular, intralesional etc.

Yet in another embodiment of the present invention, the said pharmaceutical composition is effective against all kind of species of Leishmania whether it is antimonials resistant or antimonials sensitive.

The present invention also provides a method for the preparation of liposomal formulation, wherein the said method comprising the steps of:
a) preparing a lipid film comprising a neutral and cationic lipid in a molar ratio of 7:2 respectively;
b) encapsulating the antileishmanial antimonial drugs by dispersing the lipid film obtained from step (b) in PBS solution of pH 7.4, containing said antileishmanial antimonial drugs preferably sodium antimony gluconate, wherein the ratio of the lipid to PBS solution is in the range of 63:1 to 44:1.
c) applying ultrasonication for about 1 minute on ice to the encapsulating the antileishmanial antimonial drugs in lipid film obtained from step (b);
d) keeping the liposome obtained from step (c) at 4° C. for 2 hours followed by centrifugation at 10,000 g for 30 minutes at 4° C. to get the desired liposomal formulation;
e) repeating the centrifugation step thrice to remove unencapsulated drug.

In an embodiment of the present invention, a uniform lipid film is prepared using rotary evaporator.

In another embodiment of the present invention, the PBS solution contains sodium chloride, sodium dihydrogen phosphate, disodium hydrogen phosphate in the ratio ranges from 10 mM to 20 mM.

The following abbreviations will be employed:
SAG—sodium antimony gluconate.
MLV—multilamellar vesicle.
PC—phosphatidylcholine.
SA—octadecylamine.
DRV—dehydrated rehydrated vesicle.
CTAB—cetryltrimethylammoniumbromide.
DDAB—dimethyldioctadecylammoniumbromide.
DOTAP—dioleyltrimethylammoniumpropane.
DMTAP—dimyristoyltrimethylammoniumpropane.
ePC—egg phosphatidylcholine.
hPC—hydrogenated egg phosphatidylcholine.
PBS—phosphate buffer saline.
HSPC—hydrogenated soy phosphatidylcholine.

The pentavalent or trivalent antimony containing drugs are highly effective antileishmanial drugs although their use is presently limited due to their toxicity and failure against resistant strain. We have found that minimal amount of encapsulated antimonials in combination with suboptimal amount of antileishmanial cationic liposome confers a synergistic therapeutic effect against Leishmania parasite, eliciting sterile protection, evading the problem of toxicity and resistance. Suitable lipids that may be used in the present invention include cationic lipids such as octadecylamine (SA), dimethyldioctadecylammoniumbromide (DDAB), cetryltrimethylammoniumbromide (CTAB) or dodecyltrimethylammoniumbromide (DTAB) after screening from the groups of other cationic lipids such as dioleyltrimethylammoniumpropane (DOTAP) and dimyristoyltrimethylammoniumpropane (DMTAP). Neutral lipid that can be used in combination with either of these cationic lipids for the formulation of the cationic liposome include phosphatidylcholine (PC), egg phosphatidylcholine (ePC) or hydrogenated egg phosphatidylcholine (hPC). We have found that a particular useful combination of neutral lipid to cationic lipid that can be used in our formulation is in a molar ratio of 7:2 respectively. Since cationic lipids show pronounced cytotoxicity against eukaryotic cells preferred combination of neutral lipid to cationic lipids are screened out critically so as to make it nontoxic towards host cell, preserving its leishmanicidal activity. It is contemplated by this invention to optionally include cholesterol in the liposome. Cholesterol is known to improve loading capacity of drug and also improve stability of liposome.

The antimonials containing drugs that can be formulated in accordance with the present invention are any of the antimonials containing drugs conventionally used to cure leishmaniasis. The drug most commonly used for this purpose is SAG, sold under the trade name Pentostam. Other antimonials containing drugs which are used to combat leishmaniasis can also be encapsulated in accordance with the presently invented cationic liposome are meglumine antimoniate (Glucantime), potassium antimony tartarate or urea stibamine.

Conventional methods for the encapsulation of antimonials containing drugs into liposome have resulted in the encapsulation of somewhere in the region of 2-10% of the drug present in the initial aqueous phase. Moreover, those vesicles proved to be leaky. Improvisation of the method of encapsulation heightened the encapsulation efficiency as well as the stability of the preparation. But all these formulations had several disadvantages such as the extremely large dosage volumes of the liposomal formulation have had to be injected in order to introduce a sufficient quantity of antimonial drug, required multiple dosing for complete cure and most importantly all the previous formulations failed to eradicate parasites hidden in deep seated organs like spleen and bone marrow and/or failed to elicit protection against antimonials-resistant parasite.

The liposomes that may be used in the invention include MLV or DRV but these may include other small or large unilamellar vesicles, reverse phase evaporation vesicle and MLV produced by freeze thaw technique. Herein, two methods may be used to prepare a cationic liposomal formulation, comprising drug and lipids. In one method, neutral and cationic lipid are combined in a molar ratio of 7:2 in organic solvent, the solution evaporated to a thin film and, after 12-16 hours desiccation, the film is hydrated with an aqueous solution containing the SAG. MLV are formed by agitation of the dispersion, preferably on vortex mixing. Unilamellar vesicles are formed by the application of a shearing force to an aqueous dispersion of the lipid solid phase example, sonication. Yet, in another method, neutral lipid and cationic lipid are mixed in a molar ratio of 7:2 either in absence or in presence of 2 molar ratio of cholesterol in organic solvent, a thin film is formed thereby, the film is dispersed at 54° C. followed by sonication in bath sonicator for 20 minutes at 20° C. The dispersed material is then probe sonicated for 10 minutes, at 54° C. with intermittent gap of 60 seconds. The resultant milky suspension is freeze-dried at −120° C. temperature to form dry lyophilized powder. The dry powder is reconstituted with 20 mM PBS when required.

We have found that by operating either of this way a normal milky liposomal dispersion forms in which subsequent tests show that about 35-50% of the antimonials compound initially present in the aqueous phase is encapsulated inside the cationic liposome.

Where necessary, as in MLV or DRV preparatory procedures, organic solvents may be used to solubilise lipids during cationic liposome preparation. Suitable organic solvents are those with a variety of polarities and dielectric properties, including chloroform and mixtures of chloroform and methanol in 1:1 (v/v).

Liposomes entrapped an aqueous medium enclosed by lipid bilayers. The aqueous medium, herein may be water containing salts or isotonic buffer. Example of such salts is sodium chloride and buffer is 20 mM PBS. Other buffers may include Tris-HCl (9-tris-9-hydroxymethyl-amino methane hydrochloride) or HEPES (N-2-hydroxyethyl piperazine-N′-2-ethane sulphonic acid). Buffers may be present in the pH range between 7-7.8. In the preferred embodiment, the lipid film is hydrated with 20 mM PBS at pH 7.4.

Regardless of the method used for formation of the liposome, there will be inevitably be significant amounts of antimonies that are not encapsulated into the liposome but remain in the continuous aqueous phase. For various reasons, it is often desirable to remove the drug from the continuous aqueous phase. This is conveniently done either by dialyzing the liposomal formulation against a drug free aqueous phase across a dialysis membrane or by centrifugation. Centrifugation is preferably carried out at 9,000 rpm for 30 minutes. This procedure is repeated thrice. Most of the supernatant containing unencapsulated drug is then separated from the liposomal pellet with minimum disturbance of the pellet. Liposomal pellet is finally suspended in required volume of 20 mM PBS.

The leakage of the encapsulated drug from the liposome into the continuous aqueous phase is a complicated phenomenon influenced not only by the nature and concentration of the salt present in the continuous aqueous phase but also by the amount of encapsulated drug and the nature and proportion of the lipids used for the formation of the liposome. Some leakage of encapsulated drug after liposome formation is inevitable but we have found that almost for 4 weeks, the formulations can be stored at 4° C. with leakage rates below 50%.

The antileishmanial activity of SAG entrapped cationic liposome is well studied in experimentally infected BALB/c mice model To study the antileishmanial therapy, infection of mice may be done by any Leishmania species that cause visceralization. It is also contemplated that this invention may be effective against species causing cutaneous leishmaniasis. Since antimonial drugs are first line of drug for both visceral and cutaneous leishmaniasis and our cationic liposome itself already showed to be effective against Leishmania strain causing cutaneous disease, so it can be speculated that the our liposomal antimonials must be equally effective against cutaneous form of disease.

This invention seems to be therapeutically effective against SAG-resistant parasite thereby focusing its importance during recent outbreak of resistant strain.

Herein, the referred liposomal drug is administered to infected model by intravenous route. However, when the drug need to be assisted against cutaneous leishmaniasis the formulations must be injected either intralesionally or intramuscular.

The following examples are given by way of illustration of the present invention and should not be construed to limit the scope of present invention.

### Example 1

### Preparation of Cationic Liposome and Entrapment of SAG within it

Lipids used herein were obtained as dry powder from Sigma and Fluka. SAG is bought from Gluconate Health Limited, India. All other chemicals were analytical reagent grade. A solution of lipid was prepared by dissolving 20 mg PC type X-E and 2 mg SA in approximately, 2 ml of chloroform. The molar ratio of the two lipid materials is 7:2, respectively. A uniform lipid film is made in round-bottomed flask with rotary evaporator. The lipid film is then desiccated in vacuum dessicator for almost 16 hours. For drug encapsulation, the lipid film was dispersed in 20 mM PBS, pH 7.4, containing 1 mg of SAG, and sonicated for 60 seconds in an ultrasonicator. To remove unencapsulated SAG, liposomes with entrapped SAG were washed thrice in PBS at 10,000×g, 30 min., 4° C. On measuring degree of encapsulation approximately, 30-50% of the initially added SAG was found to be associated with 22 mg of lipid.

### Example 2

### Stability Assay of Cationic Liposome

The liposomal formulation was stored at 4° C. and leakage rates of encapsulated SAG were measured after 15 and 30 days. The leakage was determined by the following way. A 1 ml suspension of liposomal formulation was placed in a polycarbonate tube with a stopper and centrifuged at 9,500×G for 30 minutes. The pellet was then suspended in 10 ml of 20 mM PBS and centrifuged thrice. The supernatants were collected in separate polypropylene tubes. The thrice-washed pellet having liposome was resuspended in 5 ml of chloroform-water mixture (1:1 v/v) and centrifuged at 14,000×G for 10 minutes, at 4° C., thrice. Supernatants were collected and then assayed for antimony level. This assay was done spectrophotometrically and the following results were obtained:

TABLE 1

| | | SAG content | | |
|---|---|---|---|---|
| | | in supernatant | | |
| Number | SAG content in | after | | Percentage |
| of days | supernatant before | chloroform | | of entrapped |
| stored. | chloroform treatment. | treatment. | Total. | SAG leaked. |
| 0 | 550 μg | 450 μg | 1000 μg | 0 |
| 15 | 600 μg | 400 μg | 1000 μg | 11.11 |
| 30 | 750 μg | 250 μg | 1000 μg | 44.44 |

This result revealed that although some leakage of encapsulated SAG occurs, substantially most of this leakage occurs at 30 days after storage and no significant leakage does occur at 15 days post storage period.

### Example 3

### In Vivo Efficacy in Established Infection Model

Inbred mice of 4-6 weeks old, weigh 20 g and of any sex, strain BALB/c were infected with Leishmania donovani, AG83, by intravenous inoculation with 2.5×10⁷ amastigotes from the spleen of an infected hamster. Eight weeks after inoculation, the mice were divided into groups of 4-5 animals and administered at a single dose intravenously into the tail vein with optimal dose of free SAG (0.3 g/kg wt.) or empty PC-SA liposome (1.1 g/kg wt.) or SAG entrapped PC-SA liposome (0.015 g/kg of SAG into 1.1 g/kg body wt.) or SAG entrapped in PC-Chol liposome (0.015 g/kg of SAG into 1.25 g/kg wt. of lipid). Mice were sacrificed on 30 days post treatment. Livers and spleens were excised and weighed. Bone marrow was also isolated from femur bone and smeared on glass slides. Impressions smears were prepared from the cut surface of the liver and spleen. The impression smears were stained with Giemsa, and number of amastigotes counted microscopically per 500 cell nuclei. The results of 30 days post treatment are shown below and expressed as percentage suppression in parasitemia with respect to untreated infected.

TABLE 2
Effect of SAG entrapped PC-SA liposome on reducing liver
parasite level of BALB/c mice infected with L. donovani AG83.
Treatment started on 8 weeks post infection at a single
shot and by intravenous route.

| | | | % Inhibition of | |
|---|---|---|---|---|
| | SAG (g/kg | PC-SA (g/kg | liver parasite | |
| Treatments. | body wt.) | body wt.) | level. | +S.E |
| Untreated | — | — | 0% | — |
| SAG | 0.3-0.4 | — | 82.322% | +0.69 |
| Blank liposome | — | 1.0-1.1 | 48.814% | +1.24 |
| Drug loaded | 0.015-0.02 | 1.0-1.1 | 98.719% | +0.059 |
| PC-SA liposome | | | | |
| Drug loaded | 0.015-0.02 | 1.25-1.30 | 12.349% | +0.438 |
| PC-Chol | | | | |
| liposome | | | | |

The results revealed that combined therapy with SAG and PC-SA liposome is better medicament than either of the monotherapies or SAG encapsulated PC-Chol liposome in controlling liver parasite burden and even its potentiality proved to be better than the optimal dose of SAG.

TABLE 3
Effect of SAG entrapped PC-SA liposome on reducing spleen
parasite level of BALB/c mice infected with L. donovani AG83.

| | | | % Inhibition of | |
|---|---|---|---|---|
| | SAG (g/kg | PC-SA (g/kg | spleen parasite | |
| Treatments. | body wt.) | body wt.) | level. | +S.E |
| Untreated | — | — | 0% | — |
| SAG | 0.3-0.4 | — | 70% | +2.167 |
| Blank liposome | — | 1.0-1.1 | 83.87% | +1.311 |
| Drug loaded PC- | 0.015-0.02 | 1.0-1.1 | 97.857% | +0.594 |
| SA liposome | | | | |
| Drug loaded PC- | 0.015-0.03 | 1.25-1.3 | 32.606% | +0.399 |
| Chol liposome | | | | |

From the above result, it seems that SAG entrapped in lipid vesicles provide better protectivity than free SAG against spleen parasite burden when compared to its efficacy against parasite having haven in liver. Even blank PC-SA liposome induce significant (p<0.001) fall in parasitemia. In contrast, optimal dose free SAG could partially effective at suppressing spleen infection.

TABLE 4
Effect of SAG entrapped PC-SA liposome on reducing bone marrow
parasite level of BALB/c mice infected with L. donovani AG83

| | SAG (g/kg | PC-SA (g/kg | % Inhibition of | |
|---|---|---|---|---|
| Treatments. | body wt.) | body wt.) | parasite level. | +S.E |
| Untreated | — | — | 0% | — |
| SAG | 0.3-0.4 | — | 44.029% | +0.09 |
| Blank liposome | — | 1.0-1.1 | 52% | +0.987 |
| Drug loaded PC- | 0.015-0.02 | 1.0-1.1 | 87.329% | +3.968 |
| SA liposome | | | | |
| Drug loaded PC- | 0.015-0.03 | 1.25-1.3 | 6.885% | +0.322 |
| Chol liposome | | | | |

Herein, the same result is resurrected against bone marrow parasites.

As the efficacy of most antileishmanial agents depend on its effect evident in spleen, liver and bone marrow, our formulation successfully exhibits almost sterile protection against liver and spleen parasite burden. Our invention also shows pronounced activity against parasite present in bone marrow. Reports are there that low numbers of parasites hidden in bone marrow, spleen or other unknown safe haven are responsible for relapse. In such regards, unlike previous report, our liposomal SAG shows promising antileishmanial activity against such deep-seated parasites.

### Example 4

### In Vivo Activity Screening in Established Infection Model to Calculate 50% Effective Dose

Inbred mice of 4-6 weeks old, weigh 20 g and of any sex, strain BALB/c were infected with Leishmania donovani, AG83, by intravenous inoculation with 2.5×10⁷ amastigotes from the spleen of an infected hamster. Eight weeks after inoculation, the mice were divided into groups of 4-5 animals and dosed intravenously into the tail vein with graded dose of free SAG, empty PC-SA liposome, or SAG entrapped PC-SA liposome. Mice were sacrificed on 30 days post treatment. The livers and spleens were excised and weighed. Impressions smears were prepared from the cut surface of the liver and spleen. The impression smears were stained with Giemsa, and number of amastigotes counted microscopically per 500 cell nuclei. The results of 30 days post treatment are expressed as percentage suppression in parasite burden in compared to infected but untreated mice. Thereby, the dosage necessary to reduce the parasite count to 50% of the untreated group could be calculated.

TABLE 05
Effect of graded dose of SAG on reducing liver and spleen
parasite level of BALB/c mice infected with L. donovani AG83

| | % Inhibition of | | % Inhibition of | |
|---|---|---|---|---|
| SAG (g/kg body | liver parasite | | spleen parasite | |
| wt.) | level. | +S.E | level. | +S.E |
| 0 | — | — | — | — |
| 0.005 | 56.55% | +0.33 | 41.27% | +0.11 |
| 0.010 | 56.7% | +0.45 | 50.95% | +0.23 |
| 0.050 | 61.29% | +0.56 | 55.67% | +0.3 |
| 0.10 | 88.03% | +0.45 | 65.79% | +0.44 |
| 0.30 | 92.73% | +0.44 | 70.49% | +1.03 |
| 0.50 | 96.6% | +0.56 | 80.33% | +0.2 |

From the above results, it seems that 0.5 g/kg body weight of SAG elicits almost 96.6% protection in liver in infected mice. But still spleen parasitemia remains quite significantly high at such highest dose.

TABLE 06
Effect of graded dose of PC-SA liposome on reducing liver and
spleen parasite level of BALB/c mice infected with L. donovani AG83

| | % Inhibition of | | % Inhibition of | |
|---|---|---|---|---|
| PC-SA (g/kg | liver parasite | | spleen parasite | |
| body wt.) | level. | +S.E | level. | +S.E |
| 0 | — | — | — | — |
| 0.55 | 32.31 | +0.22 | 27.05 | +0.56 |
| 1.1 | 48.84 | +9.24 | 83.87 | +0.33 |
| 1.65 | 78.67 | +0.33 | 84.165 | +0.45 |
| 2.75 | 93.95 | +0.23 | 85.789 | +0.56 |
| 5.5 | 96.84 | +0.33 | 96.46 | +0.33 |

In contrast, highest dose of PC-SA liposome evokes significant protection as it reduces both spleen and liver parasites level to 97%. Thus, free liposome itself is a prospective therapeutic agent.

TABLE 07
Effect of graded dose of PC-SA liposome entrapped SAG
on reducing liver and spleen parasite level of BALB/c mice
infected with L. donovani AG83

| | | % Inhibition | | | |
|---|---|---|---|---|---|
| PC-SA | | of liver | | % Inhibition | |
| (g/kg | SAG (g/kg | parasite | | of spleen | |
| body wt.) | body wt.) | level. | +S.E | parasite level. | +S.E |
| 0 | 0 | — | — | — | — |
| 0.1375 | 0.0018 | 11.1% | +0.24 | 30.54% | +0.33 |
| 0.3438 | 0.0049 | 43.51% | +0.354 | 42.41% | +0.23 |
| 0.6875 | 0.0093 | 62% | +0.56 | 75.24% | +0.23 |
| 1.1 | 0.0150 | 98.719% | +0.059 | 97.857% | +0.594 |
| 2.75 | 0.0375 | 99.7% | +0.33 | 99.99% | +0.33 |

Surprisingly, combined therapy of free liposome and conventionally used SAG synergistically enhances the therapeutic efficacy of individual therapy. 2.75 g/kg body wt of PC-SA entrapping SAG conferred sterile protection in experimentally infected mice.

TABLE 08

| | ED₅₀ (+S.E) (g/kg body wt.) | | | | ED₉₀ (+S.E) (g/kg body wt.) | | | |
|---|---|---|---|---|---|---|---|---|
| | Liver | | Spleen | | Liver | | Spleen | |
| Treatment | SAG | PC-SA | SAG | PC-SA | SAG | PC-SA | SAG | PC-SA |
| Free SAG | 0.012 + 0.004 | — | 0.145 + 0.020 | — | 0.347 + 0.005 | — | 0.493 + 0.028 | — |
| PC-SA | — | 1.126 + 0.088 | — | 0.655 + 0.003 | — | 2.027 + 0.029 | — | 1.180 + 0.054 |
| liposome | | | | | | | | |
| SAG entrapped | 0.007 + 0.0001 | 0.554 + 0.093 | 0.014 + 0.001 | 0.455 + 0.014 | 0.014 + 0.002 | 1.003 + 0.015 | 0.013 + 0.099 | 0.822 + 0.005 |
| PC-SA | | | | | | | | |
| liposome | | | | | | | | |

### Example 5

### In Vivo Activity Screening in Chronic Infection Model

Inbred mice of 4-6 weeks old, weigh 20 g and of any sex, strain BALB/c were infected with Leishmania donovani, AG83, by intravenous inoculation with 2.5×10⁷ amastigotes from the spleen of an infected hamster. Twelve weeks after inoculation, the mice were divided into groups of 4-5 animals and dosed intravenously into the tail vein with empty PC-SA liposome (1-1.1 g/kg body wt.), free SAG (0.015-0.020 g/kg body wt.) or equivalent amount of SAG entrapped in PC-SA liposome (0.015 g/kg of SAG in 1-1.1 g/kg body wt of liposome). Mice were sacrificed on 30 days post treatment. Livers and spleens were excised and weighed. Impressions smears were prepared from the cut surface of the liver and spleen. The impression smears were stained with Giemsa, and number of amastigotes counted microscopically per 500 cell nuclei. The results of 30 days post treatment are shown below and expressed percentage suppression in parasitemia with respect to untreated infected control calculated.

TABLE 09
Effect of SAG entrapped PC-SA liposome on reducing liver parasite level of BALB/c mice
infected with L. donovani AG83
Treatment started on 12 weeks post infection at a single shot and by intravenous route

| | SAG | PC-SA | % Inhibition of | | % Inhibition | |
|---|---|---|---|---|---|---|
| | (g/kg | (g/kg | liver parasite | | of spleen | |
| Treatment | body wt.) | body wt.) | level. | + S.E | parasite level. | + S.E |
| Untreated | 0 | 0 | — | — | — | — |
| SAG | 0.015-0.02 | — | 50.88% | +1.009 | 26.69% | +0.78 |
| Blank | — | 1-1.1 | 6.71% | +0.09 | 43.67% | +0.77 |
| liposome | | | | | | |
| Drug loaded | 0.015-0.02 | 1-1.1 | 98.92% | +0.98 | 96.09% | +0.065 |
| PC-SA | | | | | | |
| liposome | | | | | | |

Previous drug associated liposomal formulations are reported to be effective in infection model where visceralisation are observed till 4 weeks. In our study, efficacy of drug is judged in 12 weeks infection model were the extent of parasitemia is higher. This result focuses on its effectiveness against chronically infected mice burdened with quite high level of leishmania parasites.

### Example 6

### In Vivo Toxicity Assay

A few parameters, such as specific enzyme levels related to normal liver and kidney functions, were chosen to determine the toxic effects of drug. Analyses in serum were done at day 15 after injection of graded dose of SAG entrapped PC-SA liposome to the normal 4-6 wk old BALB/c mice. Assays were performed for serum creatinine, serum urea, serum glutamate pyruvate transaminase, serum alkaline-phosphatase levels (using diagnostic kits from Dr. Reddy's laboratories).

TABLE 10
Death report of normal BALB/c mice inoculated
with PC-SA liposome entrapping SAG

| PC-SA | SAG | Number of | Number | % of |
|---|---|---|---|---|
| (g/kg | (g/kg body | experimental | of mice | mortality |
| body wt.) | wt.) | mice taken | died | rate |
| 0 | 0 | 6 | 0 | 0% |
| 0.137 | 0.002 | 6 | 0 | 0% |
| 0.343 | 0.005 | 6 | 0 | 0% |
| 0.662 | 0.009 | 6 | 0 | 0% |
| 1.1 | 0.015 | 6 | 0 | 0% |
| 2.75 | 0.038 | 6 | 4 | 66.6% |

All other doses, except 2.75 g/kg body wt. dosage, seem to be non lethal. LD₅₀ for PC-SA-SAG is 2.5 g/kg body wt. PC-SA-SAG liposomal dose show almost 70% lethality.

TABLE 11
In vivo toxicity study with normal BALB/C mice inoculated
with SAG entrapped PC-SA liposome

| PC-SA | | | | |
|---|---|---|---|---|
| (g/kg | | | | Urea |
| body | SAG (g/kg | Creatinine | SGPT | (Mg % of urea) + |
| wt.) | body wt.) | (mg/dl) + S.E | (U/ml) + S.E | S.E |
| 0 | 0 | 0.945 + 0.021 | 25.5 + 0.289 | 34.14 + 0.185 |
| 0.137 | 0.002 | 1.297 + 0.012 | 26.83 + 0.600 | 37.12 + 0.185 |
| 0.343 | 0.005 | 1.006 + 0.037 | 22.0 + 0.577 | 40.91 + 0.116 |
| 0.662 | 0.009 | 1.005 + 0.006 | 23.5 + 0.006 | 34.5 + 00.065 |
| 1.1 | 0.015 | 1.000 + 0.97 | 25.3 + 0.0056 | 32.56 + 0.098 |
| 2.75 | 0.038 | 1.9 + 0.012 | 30.78 + 0.0043 | 48.97 + 0.056 |

Among the dosage screened, 2.75 g/kg body wt. show best therapeutic result but this being lethal as well as toxic dose, 1.1 g/kg body wt. dose is chosen to be the optimal therapeutic dose. The optimal dose is nontoxic as revealed by liver and renal toxicity assay.

### Example 7

### In Vivo Efficacy Assay Against Sag-Resistant Strain

Inbred mice of 4-6 weeks old, weigh 20 mg and of any sex, strain BALB/c were infected with Leishmania donovani GE1F8R strain by intravenous inoculation with 2.5×10⁷ amastigotes from the spleen of an infected hamster. Eight weeks after inoculation, the mice were divided into groups of 4-5 animals and dosed intravenously into the tail vein with optimal dose of free SAG, equivalent amount of free SAG, empty PC-SA liposome or SAG entrapped PC-SA liposome (Please scratch out the dose). Mice were sacrificed on 30 days post treatment. Livers and spleens were excised and weighed. Bone marrow was also isolated and smeared on glass slides. Impressions smears were prepared from the cut surface of the liver and spleen. The impression smears were stained with Giemsa, and number of amastigotes counted microscopically per 500 cell nuclei. The results of 30 days post treatment are expressed as Leishman Donovan units and percentage suppression in parasitemia with respect untreated infected control were calculated.

TABLE 12
Effect of SAG entrapped PC-SA liposome on reducing liver
parasite level of BALB/c mice infected with L. donovani GE1FT8R.

| | | PC-SA | | |
|---|---|---|---|---|
| | SAG | (g/kg | | |
| | (g/kg body | body | % Inhibition of | |
| Treatments. | wt.) | wt.) | parasite level. | +S.E |
| Untreated | 0 | 0 | 0% | — |
| SAG | 0.3 | — | 5.309% | +3.589 |
| SAG | 0.015-0.02 | — | 0% | — |
| Drug loaded PC- | 0.015-0.02 | 1-1.1 | 93.856% | +1.720 |
| SA liposome | | | | |
| Drug loaded PC- | 0.015-0.03 | 1.2-1.3 | 0% | — |
| Chol liposome | | | | |
| Amphotericin B | 0.002-0.0025 | — | 87.778% | +0.2441 |

TABLE 13
Effect of SAG entrapped PC-SA liposome on reducing spleen
parasite level of BALB/c mice infected with L. donovani GE1FT8R.

| | SAG | PC-SA | | |
|---|---|---|---|---|
| | (g/kg body | (g/kg | % Inhibition of | |
| Treatments. | wt.) | body wt.) | parasite level. | +S.E |
| Untreated | 0 | 0 | 0% | — |
| SAG | 0.3 | — | 16.836% | +8.434 |
| SAG | 0.015-0.02 | — | 0% | — |
| Drug loaded PC- | 0.015-0.02 | 1-1.1 | 97.56% | +0.809 |
| SA liposome | | | | |
| Drug loaded PC- | 0.015-0.03 | 1.2-1.3 | 0% | — |
| Chol liposome | | | | |
| Amphotericin B | 0.002-0.0025 | — | 85.091% | +2.165 |

TABLE 14
Effect of SAG entrapped PC-SA liposome on reducing bone marrow
parasite level of BALB/c mice infected with L. donovani GE1FT8R

| | SAG | PC-SA | | |
|---|---|---|---|---|
| | (g/kg body | (g/kg | % Inhibition of | |
| Treatments. | wt.) | body wt.) | parasite level. | +S.E |
| Untreated | 0 | 0 | 0% | — |
| SAG | 0.3 | — | 16.836% | +8.434 |
| SAG | 0.015-0.02 | — | 0% | — |
| Drug loaded PC- | 0.015-0.02 | 1-1.1 | 84.313% | +6.297 |
| SA liposome | | | | |
| Drug loaded PC- | 0.015-0.03 | 1.2-1.3 | 14.572% | +2.271 |
| Chol liposome | | | | |
| Amphotericin B | 0.002-0.0025 | — | 85.091% | +2.165 |

The efficacy of SAG entrapped PC-SA liposome against SAG-resistant strain is reflected to the extent of above 90% against liver and splenic parasite burden and 84% against bone marrow parasite. It reveals the importance of SAG-loaded cytotoxic liposome against SAG resistant strain.

### Advantages:

The main advantages of the present invention are:
1. The claimed cationic liposomal formulations of SAG are able to elicit almost sterile protection against liver and spleen parasite burden.
2. The claimed cationic liposomal formulations are able to confer satisfactory level of protection against deep-seated parasite in bone marrow.
3. The claimed cationic liposomal formulations of SAG provide protection against chronically infected mice.
4. The claimed cationic liposomal formulations of SAG are effective against both sensitive and resistant strains of L. donovani.
5. The claimed pharmaceutical formulations required minimal amount of SAG to contain parasite from the organs.
6. The claimed pharmaceutical formulations required minimal amount of SAG thereby avoiding unnecessary toxicity associated with free SAG.

### REFERENCES

### References by U.S. Patent Document

| | | | |
|---|---|---|---|
| 4,594,241 | June, 1986 | Rao, L. S. | 424/450 |
| 6,759,057 | July, 2004 | Weiner et al. | 424/450 |
| 3,993,754 | November, 1976 | Rahman et al. | 514/12 |
| 4,145,410 | March, 1979 | Sears | 424/450 |
| 4,235,871 | November, 1980 | Papahadjopoulos et | 424/450 |
| | | al. | |

### Other References

Berman, et. al., Recommendations for treating leishmaniasis with sodium stibogluconate (Pentostam) and review of pertinent clinical studies, 1992, Am. J. Trop. Med. Hyg, 46: 296-306.
Thakur, et. al., Do the diminishing efficacy and increasing toxicity of sodium stibogluconate in the treatment of visceral leishmaniasis in Bihar, India; justify its continued use as a first-line drug? An observational study of 80 cases, 1998, Ann. Trop. Med. Parasitol, 92: 561-569.
Jha, et. al., Evaluation of diamidine compound (pentamidine isethionate) in the treatment resistant cases of kala-azar occurring in North Bihar, India, 1983, Trans. R. Soc. Trop. Med. Hyg, 77: 167-170.
Jha, et. al., Use of amphotericin B in drug-resistant cases of visceral leishmaniasis in north Bihar, India, 1995, Am. J. Trop. Med. Hyg, 52: 536-538.
Ali, et. al., Treatment of visceral leishmaniasis with sodium stibogluconate in Sudan: management of those who do not respond, 1998, Ann. Trop. Med. Parasitol, 92: 151-158.
Bryceson, et. al., Short-course treatment of visceral leishmaniasis with liposomal amphotericin B (AmBisome), 1996, Clin. Infect. Dis, 22: 938-943.
Dey, et. al., Antileishmanial activities of stearylamine-bearing liposomes, 2000, Antimicrob. Agents. Chemother, 44: 1739-1742.
Murray, et. al., Short-course, low-dose amphotericin B lipid complex therapy for visceral leishmaniasis unresponsive to antimony, 1997, Ann. Intern. Med, 127: 133-137.
Alving, et. al., Therapy of leishmaniasis: superior efficacies of liposome-encapsulated drugs, 1978, Proc. Natl. Acad. Sci. USA, 75: 2959-2963.
Black, et. al., The use of Pentostam liposomes in the chemotherapy of experimental leishmaniasis, 1977, Trans. R. Soc. Trop. Med. Hyg, 71: 550-552.
Peters, et. al., Antileishmanial activity of antimonials entrapped in liposomes, 1978, Nature, 272: 55-56.
Afrin, et. al., Leishmanicidal activity of stearylamine-bearing liposomes in vitro, 2001, J. Parasitol, 87: 188-193.
Pal, et. al., Combination therapy using sodium antimony gluconate in stearylamine-bearing liposomes against established and chronic Leishmania donovani infection in BALB/c Mice, 2004, Antimicrob. Agents. Chemother, 48: 3591-3593.
Vemuri, et al. Preparation and characterization of liposomes as therapeutic delivery systems: a review, 1995, Pharm Acta Helv, 70: 95-111.
Bangham, et. al., Diffusion of univalent ions across the lamellae of swollen phospholipids, 1965, J. Mol. Biol, 13: 238-252.
Papahadjopoulas, et. al., The introduction of poliovirus RNA into cells via lipid vesicles (liposomes), 1979, Cell, 17:77-84.
Croft, C., Coombs, G. H. Leishmaniasis—Current Chemotherapy and recent advances in the search for novel drugs. Trends Parasitol. 2003; 19:502-8

1. A liposomal formulation useful as a leishmanicidal agent, wherein said formulation comprising a single dose of therapeutically effective amount of an antileishmanial antimonial drug encapsulated in a cationic liposome consisting of a neutral lipid and a cationic lipid in a molar ratio of 7:2 respectively, wherein molar ratio of the neutral lipid and the cationic lipid to said drug is 7:2:0.2 to 0.25.

2. A liposomal formulation as claimed in claim 1, wherein the antileishmanial antimonial drugs used is selected from the group consisting of pentavalent antimonial drugs, and trivalent antimonial drugs.

3. (canceled)

4. A liposomal formulation as claimed in claim 1, wherein the neutral lipid is phosphatidylcholine.

5. A liposomal formulation as claimed in claim 4, wherein the said phosphatidylcholine is selected from a group consisting of distearoylphosphatidylcholine, hydrogenated soy phosphotidylcholine, egg phosphatidylcholine, soy phosphatidylcholine, dimyristoylphosphatidylcholine, and dipalmitoylphosphatidylcholine.

6-7. (canceled)

8. A liposomal formulation as claimed in claim 1, wherein the cationic lipid is selected from the group consisting of octadecylamine, dimethyldioctadecylammoniumbromide, cetryltrimethylammoniumbromid, dodecyltrimethylammoniumbromide, dioleoyltrimethylammoniumpropane and dimyristoyltrimethylammoniumpropane.

9. A liposomal formulation as claimed in claim 8, wherein the the cationic lipid is octadecylamine.

10. A liposomal formulation as claimed in claim 8, wherein the cationic lipid is selected from the group consisting of dioleoyltrimethylammoniumpropane and dimyristoyltrimethylammoniumpropane.

11. A liposomal formulation as claimed in claim 8, wherein the cationic lipid is selected from the group consisting of dimethyldioctadecylammoniumbromide, cetryltrimethylammoniumbromide and dodecyltrimethylammoniumbromide.

12. A liposomal formulation as claimed in claim 8, wherein the cationic lipid is dodecyltrimethylammoniumbromide.

13. A liposomal formulation as claimed in claim 1, wherein said liposome is a multilamellar vesicle, unilamellar vesicle, dehydrated-rehydrated vesicle.

14. A liposomal formulation as claimed in claim 1, wherein said liposome is suspended in pharmaceutically acceptable carriers selected from the group consisting of: sodium chloride, sodium dihydrogen phosphate and disodium hydrogen phosphate in a concentration such that the osmolarity of the continuous aqueous phase is same as that of the human blood.

15. A liposomal formulation as claimed in claim 1, wherein said formulation is stable at a pH of 7-7.8 whereby the leakage rate of initially encapsulated said antimonial drugs are less than 50% by weight after storage for 4 weeks, at 4° C., from the day of encapsulation.

16. (canceled)

17. A pharmaceutical composition useful for the treatment of Kala azar in a subject, wherein said composition comprising the liposomal formulation as claimed in claim 1 optionally suspended in known pharmaceutically acceptable carrier.

18. A pharmaceutical composition as claimed in claim 17, wherein the pharmaceutically acceptable carriers used are selected from the group consisting of: sodium chloride, sodium dihydrogen phosphate and disodium hydrogen phosphate in a concentration such that the osmolarity of the continuous aqueous phase is same as that of the human blood.

19. A pharmaceutical composition as claimed in claim 17, wherein the dosage of the said composition is administered at a unit dose of at least 0.015 g/kg of sodium antimony gluconate (SAG) entrapped in 1-1.1 g/kg lipid.

20. A pharmaceutical composition as claimed in claim 17, wherein the administration route is selected from the group comprising of intravenous, intramuscular, intralesional etc.

21. A method of treating the kala azar in a subject, wherein said method comprising the step of administering to the subject a pharmaceutical composition comprising the therapeutically effective amount of liposomal formulation of claim 1 suspended in known pharmaceutically acceptable carrier.

22. (canceled)

23. A method as claimed in claim 21, wherein the pharmaceutically acceptable carriers used are selected from the group consisting of salts such as sodium chloride, sodium dihydrogen phosphate and disodium hydrogen phosphate in a concentration such that the osmolarity of the continuous aqueous phase is same that of the human blood.

24. A method as claimed in claim 21, wherein the dosage of the said composition is administered at a unit dose of at least 0.015 g/kg of SAG entrapped in 1-1.1 g/kg lipid.

25. A method as claimed in claim 21, wherein the administration route from the group comprising of intravenous, intramuscular, intralesional etc.

26. A method as claimed in claim 21, wherein said pharmaceutical composition is effective Leishmania species whether it is antimonials resistant or antimonials sensitive.

27. A method for the preparation of liposomal formulation as claimed in claim 1, wherein said method comprising the steps of:
a) preparing a lipid film comprising a neutral and cationic lipid in a molar ratio of 7:2 respectively;
b) encapsulating the antileishmanial antimonial drugs by dispersing the lipid film obtained from step (a) in PBS solution of pH 7.4, containing said antileishmanial antimonial wherein said antileishmanial antimonial is sodium antimony gluconate;
c) applying ultrasonication for about 1 minute on ice to the encapsulating the antileishmanial antimonial drugs in lipid film obtained from step (b);
d) keeping the liposome obtained from step (c) at 4° C. for 2 hours followed by centrifugation at 10,000 g for 30 minutes at 4° C. to get the desired liposomal formulation; and
e) centrifuging the preparation thrice to remove unencapsulated drug.

28. A method as claimed in claim 27, wherein a uniform lipid film is prepared using rotary evaporator.

29. A method as claimed in claim 27, wherein the PBS solution has a molarity ranging from 10 mM to 20 mM.
